(19) United States
(12) Patent Application Publication
(10) Pub. No.: US 2004/0138094 A1
(43) Pub. Date: Jul. 15, 2004

(54) Treatment regimes

(76) Inventors: Timothy Mark Willson, Durham, NC (US); Jennifer L Fox, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/470,886

(22) PCT Filed: Jan. 31, 2002
(86) PCT No.: PCT/US02/03017

(51) Int. Cl. A61K 31/00; G01N 33/53; G01N 33/567

(52) U.S. Cl. 514/1; 435/7.2

(57) ABSTRACT

This invention provides a method for identifying a treatment regime for a compound or composition effective in treating a PPAR mediated disease or condition. The method includes determingin a treatment regime for the compound or composition that causes expression of a surrogate market for PPAR delta activity whereby the determined dose and route of administration identifies an effective treatment protocol.

### FIELD OF THE INVENTION

The present invention is in the field of pharmacology and medicine. More particularly this invention relates to methods of identifying suitable treatment regimes for diseases or conditions mediated by Peroxisome Proliferator-Activated Receptors (PPARs), particularly those mediated by PPAR delta. In another aspect the invention provides a method of promoting reverse cholesterol transport in an individual.

### BACKGROUND OF THE INVENTION

In humans, lipid homestasis is a delicate balance between dietary intake, de novo synthesis and catabolism. The increased incidence of cardiovascular disease in Westernized nations has been linked to dyslipidemias associated with changes in the fat content of the diet (1). Obesity, insulin resistance and hypertension are comorbidities with these lipid disorders, which together are known as the metabolic syndrome X (2). Individuals with this condition have raised serum triglycerides and abnormally low levels of high density lipoprotein cholesterol (HDLc) (2, 3). This lipid profile is accompanied by an increase in the proportion of small-dense low density lipoprotein particles, which are prone to accumulate in the arterial wall leading to the formation of atherosclerotic cholesterol-laden foam cells (4). HDL plays a protective role through the process of “reverse cholesterol transport” whereby cholesterol is removed from peripheral cells, including the macrophage-derived foam cells, and returned to the liver (5). Agents that raise the levels of HDL by stimulating reverse cholesterol transport would provide a new therapeutic option for the prevention of atherosclerotic cardiovascular disease (6).

Recently, the ATP-binding cassette A1 (ABCA1) protein has been identified as a regulator of cholesterol and phospholipid transport from cells (7). Patients with Tangier disease or familial hypoalphalipoproteinia have been identified with loss of function mutations in the ABCA1 gene (8-10). These patients have low levels of HDLc and high triglycerides and show an increased incidence of cardiovascular disease (11). Thus, therapies that increase the expression of ABCA1 could provide a new approach to treating atherogenic dyslipidemia (5).

Fibrates are a class of drugs that have been used for decades for their beneficial effects on serum lipids. Although fibrates are predominantly triglyceride-lowering drugs that only modestly raise HDLc (12, 13), clinical trials have shown that they lower the incidence of atherosclerosis and coronary artery disease in patients with normal levels of LDLc (12, 14). Most fibrate drugs are only weakly active on human PPARα and show low selectivity over human PPARδ and PPARγ (15).

It was recently reported that the experimental fibrate drug Wy14,643 induces ABCA1 expression and cholesterol efflux from macrophages (16). However, at the concentrations employed in the study (50 μM), Wy14,643 has significant PPARδ activity [EC₅₀=35 μM for human PPARδ (15)]. Using compounds that are selective for each of the three PPAR subtypes, we have now shown that their relative ability to induce ABCA1 expression is PPARδ>PPARγ>PPARα. These data argue that the reported effects (16) of high doses of fibrates on cholesterol efflux are mediated primarily through PPARδ.

Compounds that agonise the function of human PPAR delta have been described in WO 01/00603 and have been shown to increase HDLc

The present inventors have now provided evidence that PPARδ increases cholesterol efflux from cells through an increase in the expression of the ABCA1 cholesterol and phospholipid transporter. These data and its broad tissue distribution suggest that PPARδ is an important regulator of reverse cholesterol transport in mammals, and thus has unique pharmacology that distinguishes it from the other PPAR subtypes. PPARδ modulators may therefore provide a new approach to the treatment of cardiovascular disease by promoting reverse cholesterol transport, an effect not previously associated with PPAR delta.

It would be desirable to test the effectiveness of drugs for PPAR delta mediated diseases or conditions. Measuring blood HDLc levels may indicate HDLc is increased but will not indicate the mechanism by which this occurs and thus whether the HDLc increase is clinically beneficial. Thus ABCA1 may be used as a surrogate marker for measuring activity of PPAR delta and therefore indicating the level of reverse cholesterol transport as well as increase in HDLc. This would provide a more objective standard and a test by which a drug could be tested for its effectiveness in individuals.

### SUMMARY OF THE INVENTION

Accordingly the present invention provides a method of increasing ABCA1 activity and/or expression and promoting reverse cholesterol transport in a subject, comprising administering to the subject a therapeutically effective amount of an modulator of PPAR delta.

In a further aspect, the present invention provides the use of a PPAR delta modulator for the manufacture of a medicament for the purpose of increasing ABCA1 activity and/or expression and promoting reverse cholesterol transport in a subject.

This invention also provides a method for testing a compound or composition for the treatment of a PPAR mediated disease or condition. The method comprises administering a PPAR delta modulator against one or more subjects and determining the level of ABCA1 expression and/or activity. This method is also useful in determining the level of efficacy of a treatment with a PPAR delta modulator, depending upon the extent to which the treatment can cause ABCA1 expression and/or activity. The present invention extends to the use of a compound or composition identified by this method in the manufacture of a medicament for the treatment of PPAR mediated diseases or conditions in a patient in need thereof and a method of treatment of PPAR mediated diseases or conditions which comprises the administration of a therapeutically effective amount of a compound identified by the above method to a patient in need thereof.

In a further aspect, the invention also provides a method for identifying a suitable treatment regime for treating a PPAR mediated disease or condition in an individual. The method involves determining a regime for a compound or composition which increases ABCA1 expression and/or activity comprising administering a compound, measuring ABCA1 expression and/or activity whereby the determined regime provides an effective treatment.

### DESCRIPTION OF THE DRAWINGS

FIG. 1. Regulation of ABCA1 expression and cholesterol efflux from THP1 macrophages. Compounds were used at the following concentrations: PPARδ (GW501516), 100 nM; PPARα (GW7647), 100 nM; PPARγ (GW7845), 100 nM. Data are presented as the mean of assays performed in triplicate ±S. D A. ABCA1 mRNA levels determined by RTQ-PCR. B. ApoA1-specific cholesterol efflux.

### DETAILED DESCRIPTION OF THE INVENTION

Measuring ABCA1 activity as a surrogate marker for PPAR delta activity is useful for identifying effective treatment regimes for individuals suffering from or pre disposed to diseases or conditions mediated by PPARs. hPPAR mediated diseases or conditions include dyslipidemia including associated diabetic dyslipidemia and mixed dyslipidemia, syndrome X (as defined in this application this embraces metabolic syndrome), heart failure, hypercholesteremia, cardiovascular disease including atherosclerosis, arteriosclerosis, and hypertriglyceridemia, type 11 diabetes mellitus, type I diabetes, insulin resistance, hyperlipidemia, inflammation, epithelial hyperproliferative diseases including eczema and psoriasis and conditions associated with the lung and gut and regulation of appetite and food intake in subjects suffering from disorders such as obesity, anorexia bulimia, and anorexia nervosa. Particular diseases include diabetes and cardiovascular diseases and conditions including atherosclerosis, arteriosclerosis, hypertriglyceridemia, and mixed dyslipidaemia.

The methods of this invention employing ABCA1 as a surrogate marker can be used at any stage, including during clinical trial or after approval of a pharmaceutical product by the appropriate regulatory authority (e.g. the United States Food and Drug Administration). In one embodiment, the pharmaceutical product comprises a PPAR delta modulator that is not known for effectiveness in PPAR mediated diseases or that has not been approved for use in PPAR mediated diseases. However, PPAR delta modulating compounds or compositions that already have been approved can be tested according to the invention also.

The treatment regime can include any factors relating to the treatment, including, without limitation, the identity of the drug or drugs administered, the amount administered, the timing of delivery, if the regime involves a combination of one or more drugs, the ratio of drugs in combination and their interaction, the route of delivery, and the pharmaceutical formulation employed.

The method can be performed on groups of individuals or on a single individual. Performing the method on a single individual, especially one who suffers from a PPAR mediated disease or condition, is useful in order to determine a customised treatment for protocol for the individual. Groups of individuals are useful in order to obtain data for statistical analysis in order to identify treatment protocols for a population in general. The groups of individuals can be individuals who suffer from a PPAR mediated disease or condition or alternatively, because the methods use surrogate markers, the subjects can be persons who do not suffer from a PPAR mediated disease or condition. The number of persons in a study is at the discretion of the researcher, who normally will use standard protocols for obtaining statistically significant results. Upon determining that a drug or drug combination causes expression and/or activation of ABCA1, the protocol can be supplied as a label on a package containing the drug and the protocol can be administered to a patient.

“Composition” refers to a composition of at least one drug to be tested. Accordingly, the composition can be a mixture of more than one drug provided in various ratios. The composition generally will be administered in the form of a composition comprising at least two drugs in a particular ratio. In one embodiment, the invention involves testing different ratios of various drugs to identify ratios that cause an inhibition of a reflux inhibition of a surrogate market. Alternatively, drugs can be administered separately in different timing schedules.

The drugs tested generally will be PPAR delta modulators. Other agents, which may not themselves display efficacy but which potentiate the effacy of other agents may also be used. Preferably, the drugs are hPPARδ agonists. As used herein, by “agonist”, or “activating compound”, or “activator”, or the like, is meant those compounds which have a pKi of at least 6.0, preferably at least 7.0, to the relevant PPAR, for example hPPARδ in the binding assay described in WO01/00603, and which achieve at least 50% activation of the relevant PPAR relative to the appropriate indicated positive control in the transfection assay described at concentrations of 10⁻⁵ M or less. Preferably, the agonist of this invention achieve 50% activation of human PPARδ in the transfection assay at concentrations of 10⁻⁷ M or less, more preferably 10⁻⁹M or less.

Most preferably, the drugs are selective hPPARδ agonists. As used herein, a “selective hPPARδ agonist” is a hPPARδ agonist whose EC50 for PPARδ is at least 10 fold lower than its EC50 for PPARγ and PPARα. Such selective compounds may be referred to as “10-fold selective.” EC50 is defined in the transfection assay described in WO01/00603 and is the concentration at which a compound achieves 50% of its maximum activity. Most preferred compounds are greater than 100-fold selective hPPARδ agonists.

According to the method of this invention, the therapeutic protocol is tested as a function of its ability to cause PPAR delta activation. Because there is no simple way to directly measure PPAR delta agonist activity, this invention relies on the use of ABCA1 as a surrogate marker. A “surrogate marker” for activity of PPAR delta is a marker that indicates increased or decreased activity of PPAR delta.

ABCA1 activity is easy to measure, ABCA1 expression and/or activity is a preferred surrogate marker for this invention. This can be measured under many different conditions. These include, e.g., ABCA1mRNA levels in blood or tissues using Taq Man, Quantigene or Northern blot. ABCA1 protein levels could be measured by using a specific antibody and ELISA.

Effectiveness of the treatment generally will coincide with the time ABCA1 expression and/or activity occurs, indicating reverse cholesterol transport. Therefore, it is useful to test for activity over the course of minutes or hours after administration of the drug. However, detecting increased ABCA1 expression and/or activity at any time after administration of the drug is a positive indication of efficacy.

Two important factors that can effect efficacy of a drug are dose and route of administration. For example, a certain amount of a drug usually is necessary to obtain any pharmacological effect. Overdoses of a drug can have harmful side effects. Similarly, because it effects bioavailability, among other things, routes of administration also effect efficacy. Thus, this invention contemplates determining the effect of dose or route of administration, alone or together, as part of determining a ABCA1 activity formulation and dosing regime.

The dose administered will be a dose within a range determined to be safe. For example, dosage amounts will be between about 0.02-5000 mg per day.

The composition can be formulated for administration in a variety of ways. Typical routes of administration include both enteral and parenteral. These include, without limitation, subcutaneous, intramuscular, intravenous, intraperitoneal, intramedullary, intrapericardiac, intrabursal, oral, sublingual, ocular, nasal, topical, transdermal, transmucosal, or anal. The mode of administration can be, eg. via swallowing, inhalation, injection or topical application to a surface (e.g., eyes, mucus membrane, skin). The formulation can be in any of the usual forms, including aqueous solution, for enteral, parenteral or transmucosal administration, e.g., for intravenous administration, as liquid formulations and administration to mucus or other membranes as, for example, nose (nasal) or eye drops; solid and other non-aqueous compositions for enteral or transdermal delivery e.g., as pills, tablets, powders, ointments, suppositories or capsules; transdermal, transmucosal or rectal delivery systems for topical administration, and aerosols or mists for delivery by inhalation.

Another parameter of the treatment regime can be timing of administration, e.g., time between doses, timed release of doses, etc.

Testing may indicate that treatment regime results in no change in the surrogate marker, an inhibition of the marker or a stimulation of the marker. Those that cause increase in activity of the surrogate marker within a safe range indicate useful protocols for delivery of the drug for therapeutic treatment.

This invention allows one to determine level of efficacy of a treatment. Compounds known to be effective in the treatment of PPAR mediated diseases can be tested to determine the level of ABCA1 activity and a standard set up relating amount of ABCA1 activity with amount of efficacy. Then, a composition can be tested in a treatment regime and the level of ABCA1 activity measured. This level is then compared with the standard curve to determine the expected level of efficacy.

### EXAMPLES

The invention will now be illustrated by reference to the following examples which should not be construed as a limitation thereto.

Cell Culture and ABCA1 Expression

THP1 human monocyte cells (ATCC TID-202), in 6-well plates at a density of 1×10⁶ cells/well in RPMI 1640 medium containing 10% FBS, were differentiated into macrophages by treatment with PMA (100 ng/ml) for 5 days. 1BR3N human skin fibroblasts (ECACC 90020508) and FHS74 human intestinal cells (ATCC CCL-241) were plated in 6 well plates at a density of 1×10⁶ cells/well in the recommended maintenance media. Cells were treated with vehicle (0.1% DMSO) or test compound for 48 h prior to harvesting. Total RNA was generated using the Qiagen RNeasy Mini Kit and DNase treated according to the manufacturer's protocol (Ambion). ABCA1 expression was analysed using RTQ-PCR on an ABI PRISM 7700 sequence detection system (P. E. Applied Biosystems). Primer/probe sequences used were as follows: ABCA1 forward primer 5′-TGTCCAGTCCAGTMTGGTTCTGTGT-3′, (Seq ID No:1) reverse primer 5′-GCGAGATATGGTCCGGATTG-3′, (Seq ID No:2) probe 5′FAM-ACACCTGGAGAGAAGCTTTCMCGAGACTMCC-TAMRA3′ (Seq ID No:3) Expression data were normalised to 18S as described by the vendor.

Cholesterol Efflux Studies

Following differentiation to macrophages, THP1 cells were washed in serum-free medium and incubated in 5% FBS medium containing 1 μCi/mL of [³H]-cholesterol (New England Nuclear) and 0.2% fatty acid-free BSA (faf BSA) for a further 24 h. The cells were washed in serum-free medium and then incubated for 24 h in serum-free medium supplemented with 1.5% faf BSA±test compound or vehicle (0.1% DMSO). The equilibration medium was removed and the cells washed twice in serum-free medium. Serum-free DMEM containing compound±purified human apolipoprotein (apo) Al (10 mg/ml, Athens Research & Technology) was added. The cells were maintained for 24 h before medium and cell extract samples were generated. Scintillation counting was performed to determine the percentage cellular cholesterol efflux±apoAl. 1BR3N human skin fibroblast cells were plated in 6-well plates at a density of 1×10⁶ cells/well in DMEM medium containing 10% FBS and cultured for 24 h. Efflux studies were performed as in the THP1 macrophages except that the cells were maintained for 48 h before scintillation counting was performed.

### Example 1

Recently, several nuclear receptors that form heterodimers with RXR have been evaluated for their ability to regulate expression of the cholesterol transporter ABCA1. Repa, J. J., Turley, S. D., Lobaccaro, J. M. A., Medina, J., Li, L., Lustig, K., Shan, B., Heyman, R. A., Dietschy, J. M. & Mangelsdorf, D. J. (2000) Science 289, 1524-1529) for either RXR or the liver X receptor α (LXRα) induce ABCA1 expression in murine peritoneal macrophages, human primary macrophages or THP1 macrophages (Repa, J. J., Turley, S. D., Lobaccaro, J. M. A., Medina, J., Li, L., Lustig, K., Shan, B., Heyman, R. A., Dietschy, J. M. & Mangelsdorf, D. J. (2000) Science 289,1524-1529. Venkateswaran, A., Laffifte, B. A., Joseph, S. B., Mak, P. A., Wilpitz, D. C., Edwards, P. A. & Tontonoz, P. (2000) Proc. Natl. Acad. Sci. USA 97, 12097-12102. Costet, P., Luo, Y., Wang, N. & Tall, A. R. (2000) J. Biol. Chem. 275, 28240-28245).

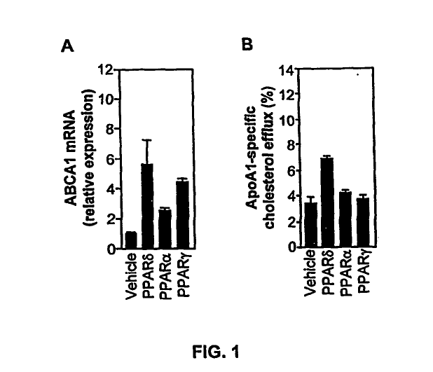

To assess the potential for the PPARs to regulate this process, THP1 macrophages were dosed with ligands selective for each of the three subtypes. At 100 nM, GW7647 (Willson T. M et al (2000) J. Med. Chem. 43 527-550, Brown P. J. et al (1997) Chem Bio. 4, 909-918) is a selective PPARα agonist (EC₅₀=6, 1070, and 6200 nM on human PPARα, PPARγ, and PPARδ, respectively), GW7845 (Willson T. M. et al (2000) J. Med. Chem. 43 527-550, Cobb, J. E. et al (1998) J. Med Chem 41 5055-5069) is a selective PPARγ agonist (EC₅₀=3500, 0.7, and >10000 nM on human PPARα, PPARγ, and PPARδ respectively), and GW501516 (2-(2-methyl-4[({4-methyl-2[4-(trifluoromethyl)phenyl]-1,3-thiazol-5-yl}methyl)sulfanyl]phenoxy}acetic acid, WO01/00603) is a selective PPARδ agonist (EC₅₀=1400, 1200, and 1.2 nM on human PPARα, PPARγ, and PPARδ, respectively). The PPARδ agonist GW501516 showed strong induction of ABCA1 mRNA expression (FIG. 1A). The PPARγ agonist GW7845 also induced ABCA1 expression, while the PPARα agonist GW7647 showed only a weak effect. The PPARδ agonist GW501516 produced a 2-fold increase in cholesterol efflux to apoAl (FIG. 1B and Table 1). Thus, PPARδ regulates cholesterol efflux from macrophages. By contrast, both the PPARα and PPARγ agonists were ineffective, despite their ability to produce small increases in ABCA1 expression in these cells.

TABLE 1
Effect of GW501516 on ABCA1
expression and cholesterol efflux.

| | | ABCA1 | Cholesterol |
|---|---|---|---|
| | Cell | mRNA (% | efflux (% |
| Cell line | Type | increase) | increase) |
| THP1 | Macrophage | 460 ± 150 | 100 ± 15 |
| 1BR3N | Fibroblast | 240 ± 25 | 110 ± 10 |
| FHS74 | Intestinal | 115 ± 30 | N. D. |

Percent increase was calculated from (GW-vehicle)/vehicle × 100. Data are presented as the mean of assays performed in triplicate ± S. D.
N. D., not determined.

### Example 2

PPARδ is expressed in many tissues that contribute to cholesterol flux (22). To evaluate whether PPARδ plays a broad role in the regulation of reverse cholesterol transport, we evaluated GW501516 in human fibroblast and intestinal cell lines (Table 1). In 1BR3N human skin fibroblasts, the selective PPARδ agonist produced a 3.4-fold increase in ABCA1 expression and a 2-fold increase in apoA1-specific cholesterol efflux. In FHS74 human intestinal cells (23), the selective PPARδ agonist produced a 2.1-fold increase in ABCA1 expression. These results, combined with the effect of GW501516 in macrophages, suggest that PPARδ agonists may promote reverse cholesterol transport from multiple tissues.

### REFERENCES

1. Lichtenstein, A. H., Kennedy, E., Barrier, P., Ernst, N. D., Grundy, S. M., Leveille, G. A., Van Home, L., Williams, C. L. & Booth, S. L. (1998) Nutr. Rev. 56, S3-S28.

2. Grundy, S. M. (1998) Am. J. Cardiol. 81, 18B-25B.

3. Ginsberg, H. N. (2000) J. Clin. Invest 106, 453458.

4. Marais, A. D. (2000) Curr. Opin. Lipidol. 11, 597-602.

5. Tall, A. R. & Wang, N. (2000) J. Clin. Invest. 106, 1205-1207.

6. von Eckardstein, A. & Assmann, G. (2000) Curr. Opin. Lipidol. 11, 627-637.

7. Schmitz, G., Kaminski, W. E. & Orso, E. (2000) Curr. Opin. Lipidol. 11, 493-501.

8. Brooks-Wilson, A., Marcil, M., Clee, S. M., Zhang, L.-H., Roomp, K., Van Dam, M., Yu, L., Brewer, C., Collins, J. A., Molhuizen, H. O. F., et al. (1999) Nat. Genet. 22, 336-345.

9. Bodzioch, M., Orso, E., Klucken, J., Langmann, T., Bottcher, A., Diederich, W., Drobnik, W., Barlage, S., Buchler, C., Porsch-Ozcurumez, M., et al. (1999) Nat. Genet. 22, 347-351.

10. Rust, S., Rosier, M., Funke, H., Real, J., Amoura, Z., Piette, J.-C., Deleuze, J.-F., Brewer, H. B., Duverger, N., Denefle, P., et al. (1999) Nat Genet 22, 352-355.

11. Clee, S. M., Kastelein, J. J. P., Dam, M. v., Marcil, M., Roomp, K., Zwarts, K. Y., Collins, J. A., Roelants, R., Tamasawa, N., Stulc, T., et al. (2000) J. Clin. Invest. 106, 1263-1270.

12. Staels, B., Dallongeville, J., Auwerx, J., Schoonjans, K., Leitersdorf, E. & Fruchart, J.-C. (1998) Circulation 98, 2088-2093.

13. Hansen, B. C. (2000) in Obesity: pathology and therapy, eds. Lockwood, D. H. & Heffner, T. G. (Springer-Verlag, Berlin), pp. 461489.

14. Rubins, H. B., Robins, S. J., Collins, D., Fye, C. L., Anderson, J. W., Elam, M. B., Faas, F. H., Linares, E., Schaefer, E. J., Schectman, G., et al. (1999) N. Engl. J. Med. 341,410418.

15. Willson, T. M., Brown, P. J., Stembach, D. D. & Henke, B. R. (2000) J. Med. Chem. 43, 527-550.

16. Chinetti, G., Lestavel, S., Bocher, V., Remaley, A. T., Neve, B., Torra, I. P., Teissier, E., Minnich, A., Jaye, M., Duverger, N., et al., (2001) Nat. Med. 7, 53-58.

17. Repa, J. J., Turley, S. D., Lobaccaro, J. M. A., Medina, J., Li, L., Lustig, K., Shan, B., Heyman, R. A., Dietschy, J. M. & Mangelsdorf, D. J. (2000) Science 289, 1524-1529.

18. Venkateswaran, A., Laffitte, B. A., Joseph, S. B., Mak, P. A., Wilpitz, D. C., Edwards, P. A. & Tontonoz, P. (2000) Proc. Natl. Acad. Sci. USA 97, 12097-12102.

19. Costet, P., Luo, Y., Wang, N. & Tall, A. R. (2000) J. Biol. Chem. 275, 28240-28245.

20. Brown, P. J., Smith-Oliver, T. A., Charifson, P. S., Tomkinson, N. C. O., Fivush, A. M., Sternbach, D. D., Wade, L. E., Orband-Miller, L., Parks, D. J., Blanchard, S. G., et al. (1997) Chem. Biol. 4, 909-918.

21. Cobb, J. E., Blanchard, S. G., Boswell, E. G., Brown, K. K., Charifson, P. S., Cooper, J. P., Collins, J. L., Dezube, M., Henke, B. R., Hull-Ryde, E. A., et al. (1998) J. Med. Chem. 41, 5055-5069.

22. Braissant, O., Foufelle, F., Scotto, C., Dauca, M. & Wahli, W. (1996) Endocrinology 137, 354-66.

23. Owens, R. B., Smith, H. S., Nelson-Rees, W. A. & Springer, E. L. (1976) J. Natl. Cancer Inst. 56, 843-849.

1. A method of increasing ABCA1 activity and/or expression and promoting reverse cholesterol transport in a subject, comprising administering to the subject a therapeutically effective amount of an modulator of PPAR delta.

2. Use of a PPAR delta modulator for the manufacture of a medicament for the purpose of increasing ABCA1 activity and/or expression and promoting reverse cholesterol transport in a subject.

3. A method for testing a compound or composition for the treatment of a PPAR mediated disease or condition comprising administering a PPAR delta modulator against one or more subjects and determining the level of ABCA1 activity and/or expression.

4. A method of treatment of PPAR mediated diseases or conditions which comprises the administration of a therapeutically effective amount of a compound identified by the method of claim 3 to a patient in need thereof.

5. A method for identifying a suitable treatment regime for treating a PPAR mediated disease or condition in an individual comprising determining a regime for a compound or composition which increases ABCA1 activity and/or expression comprising administering a compound, measuring ABCA1 activity and/or expression whereby trhe determined regime provides an effective treatment.


SEQUENCE LISTING (3 sequences)

SEQ ID NO: 1 - DNA, 26 bp, Artificial Sequence
  ATP-binding cassette A1 (ABCA1) forward primer
tgtccagtccagtaatggttctgtgt

SEQ ID NO: 2 - DNA, 20 bp, Artificial Sequence
  ATP-binding cassette A1 (ABCA1) reverse primer
gcgagatatggtccggattg

SEQ ID NO: 3 - DNA, 33 bp, Artificial Sequence
  ATP-binding cassette A1(ABCA1) probe
acacctggagagaagctttcaacgagactaacc